(19) United States
(12) Patent Application Publication
(10) Pub. No.: US 2010/0009341 A1
(43) Pub. Date: Jan. 14, 2010

(54) METHODS AND MEANS FOR ASSESSING HIV GAG/PROTEASE INHIBITOR THERAPY

(76) Inventors: Inky Paul Madeleine De Baere, Temse (BE); Guenter Kraus, Sint-Katelijne-Waver (BE); Laurence Tatiana Ramsky, Kapellen (BE); Bart Anna Julien Maes, Rumst (BE); Hilde Azijn, Leuven (BE); Marie-Pierre T.M.M.G. De Bethune, Everberg (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/296,987

(22) PCT Filed: Apr. 13, 2007
(86) PCT No.: PCT/EP2007/053613

§ 371 Date: Oct. 13, 2008

(30) Foreign Application Priority Data
Apr. 14, 2006 (EP) 06112680

(51) Int. Cl. C12Q 1/70; C07H 21/00; C12N 15/63; C12P 19/34; C12Q 1/68; C12N 7/00

(52) U.S. Cl. 435/5; 536/24.33; 435/320.1; 435/91.2; 435/6; 536/23.2; 435/235.1

(57) ABSTRACT

The present invention relates to methods and means for the evaluation of HIV treatment. In particular, molecular events at the HIV gag and protease proteins and their effect on therapeutic efficacy of drugs are determined The methods rely on providing HIV gag and protease nucleic acid material and evaluating a treatment either through genotyping or phenotyping. Said method may find use in multiple fields including diagnostics, drug screening, pharmacogenetics and drug development.

### CROSS REFERENCE TO RELATED APPLICATIONS

This application claims priority of the benefits of the filing of EP Application No. EP/06112680.1 filed Apr. 14, 2006, and PCT Application No. PCT/EP2007/053613 filed Apr. 13, 2007. The complete disclosures of the aforementioned related patent applications are hereby incorporated herein by reference for all purposes.

The present invention relates to methods and means for the evaluation of HIV treatment. In particular, molecular events at the HIV gag and protease proteins and their effect on therapeutic efficacy of drugs are determined The methods rely on providing HIV gag and protease nucleic acid material and evaluating a treatment either through genotyping or phenotyping. Said method may find use in multiple fields including diagnostics, drug screening, pharmacogenetics and drug development.

Combination drug regimens consisting of reverse transcriptase (RT) and protease inhibitors (PIs) have proven to be highly effective in suppressing human immunodeficiency virus (HIV) replication for a sustained period of time (Carpenter et al. 2000, JAMA). However, the effectiveness of these therapies is often blunted after the emergence of drug-resistant viruses, which frequently show extensive cross-resistance within each drug class (Deeks, S, 2001, J. Acquir. Immune Defic. Syndr; De Mendoza et al., 2000, J. Acquir. Immune Defic. Syndr.; Loveday, C, 2001, J. Acquir. Immune Defic. Syndr.; Miller, V, 2001, J. Acquir. Immune Defic. Syndr.).

In particular, an accumulation over time of primary and secondary amino acid substitutions in the protease enzyme has been reported in PI-resistant viruses (Condra et al., 1994, Nature; Molla et al., 1996, Nat. Med.). Some of the secondary variations found in mutant proteases also occur in HIV-1 isolates from patients who never received PI treatment. Non-subtype B proteases often display a number of such sequence deviations, leading to altered enzyme inhibition characteristics with currently available inhibitors (Kozal et al., 1996, Nat. Med.; Lech et al., 1996, J. Virol.; Shafer et al., 1999, J. Virol.; Velasquez-Campoy et al., 2001, Proc. Natl. Acad. Sci.; Vergne et al., 1998, J. Clin. Microbiol.).

It has been observed that drug-resistant mutants that have the ability to replicate need an orchestrated cleavage at the different gag, gag-pol and nef recognition sites (Henderson et al., 1992, J. Virol.; Lightfoote et al., 1986, J. Virol.; Pettit et al., 1993, Perspect. Drug Discov. Des.; Veronese et al., 1987, AIDS Res. Hum. Retroviruses; Welker et al., 1996, Virology; Freund et al., 1994, Eur. J. Biochem.). This cleavage is performed by the protease enzyme. It has also been reported the presence of mutations, insertions and deletions at multiple cleavage sites (CS) of the gag, gag-pol and nef regions in PI-resistant variants. However, only the effects of CS alterations at the gag p7/p1 and pl/p6 sites have been the subject of detailed investigations (Cote et al., 2001, J. Virol.; Doyon et al., 1996, J. Virol.; Zhang et al., 1997, J. Virol.).

Maguire et al. (Journal of Virology, 2002) have provided evidence to support that mutations in the gag region are related with increased resistance to PIs. In particular, Maguire et al. reported that changes at gag positions 449 and 453 can lead to significant decreases in susceptibility to amprenavir.

From another perspective, Gatanaga et al.(Journal of Biological Chemistry, 2002) have reported that amino acid substitutions in the gag protein at non-cleavage sites are indispensable for the development of a high multitude of HIV-1 resistance against protease inhibitors. Strikingly, Gatanaga et al. went further in concluding that non-cleavage site amino acid substitutions in the gag protein recover the reduced replicative fitness of HIV-1 caused by mutations in the viral protease. In other words, Gatanaga et al. suggest that the loss of viral fitness due to protease mutations can be overcome by mutations in the gag region.

In addition to the mutations resulting in amino acid substitutions described above, mutations causing a ribosomal frameshift might also influence resistance to viral protease inhibitors (as described by Girnary et al., Journal of General Virology, 2007, 88: 226-235).

From the foregoing, it can be affirmed that the complex interactions between the gag and protease sequence regions and the expressing products thereof, as well as any alteration within these adjacent regions, have an effect on viral fitness and drug resistance. As such a method, which can study both gag and protease regions together and in their entirety, is a desired goal. Moreover, the study of the effects of existing protease inhibitors as well as the development of new antivirals which interact at the gag or gag-pol regions—such as gag processing inhibitors (or maturation inhibitors), capsid protein polymerization inhibitors, budding inhibitors or assembly inhibitors-, increase the demand for such methods.

This objective does not come free of burdens. The gag-protease sequence is known to have a big size (approximately 1.8 Kb) which jeopardizes the generation of a full gag-protease amplified sequence (also named herein as “amplicon”). Furthermore, the gag region has a secondary structure which requires special polymerase chain reaction (PCR) conditions to achieve an optimal amplification and sequencing. Importantly, the gag region has variable base additions and deletions, and added to the fact that the protease genetic sequence is also highly variable, it increases the difficulty in obtaining a gag-protease amplicon.

Moreover, in the creation of suitable vectors carrying deletions of the gag and protease genes, it has been observed empirically that such vectors or plasmids lack stability.

WO02/20852 discloses sequences of nucleic acid oligonucleotides for amplifying different portions of gag and pol genes of HIV-1 and for detecting such amplified nucleic acid sequences. WO02/20852 further provides methods of amplifying and detecting HIV-1 nucleic acid in a biological sample using the amplification oligonucleotides specific for the gag and pol target sequences.

US20040038199 relates to methods for generating recombinant viruses from samples such as uncharacterised virus samples or clinical specimens and to the use of the viruses so generated in phenotyping assays for the purpose of detecting altered viral susceptibility to anti-viral drugs and reagents. In particular, the sequence derived from the clinical isolate includes the protease and the entire gag sequence. The method requires the performance of 2 PCRs in order to generate the whole sequence of the virus: one PCR to amplify the HIV region encompassed between the 5′ LTR promoter and the integrase sequences, and a second PCR to amplify the region ranging from the vif and the promoter 3′ LTR sequences. The two generated amplicons consist of 4 and 5 kb. The method is followed by recombination of these two constructs which recombination consists of overlapping one common region.

Due to the fidelity constraints of the polymerization enzymes and methods currently available, the amplification of sequences of big size, such as 4 and 5 kb, is not desirable. In addition, the need of a high success rate in recombination experiments advises against overlapping one common region only.

WO02/038792 concerns a method for analyzing phenotypic characteristics exhibited by certain virus strains, in particular, the human immunodeficiency viruses, using the construct of a recombinant virus obtained by homologous recombination. There is also provided a kit comprising primers, vectors, cell hosts, products and reagents necessary for producing PCR amplification, and the products and reagents for detecting a marker. In particular, the method of WO02/038792 consists of the recombination of one linearised plasmid, one circular plasmid, and one amplicon. The linearised plasmid encompasses the whole HIV genome except for the RT and the env genes, the second (circular) plasmid encompasses the env gene which is expressed by the CMV promoter. The RT sequence is amplified by the primers provided therein.

WO05/108606 relates to a method of analysing a sample containing an HIV virus. The method comprises the steps of viral RNA extraction; RNA inverse transcription and amplification thereof with a first primer pair; sequencing of the amplified reverse transcription product; amplification of the amplified reverse transcription product with a second primer pair; homologous recombination of the amplification product with a vector; functional analysis of viral proteins encoded by all or part of at least two genes; and measurement of the replicative capacity of the recombinant viruses thereby obtained. In an embodiment of WO05/108606, there is provided the analysis of a portion of the gag-pol region. In particular, the amplification product encompasses only part of the p17 sequence of the gag (i.e. starting at the base position 1165 of p17) and the adjacent full protease region. They elect to recombine this portion with a vector encompassing the backbone of HIV and the missing p17 portion, which is a wild-type sequence. As a consequence, the generated recombinant virus is not useful in testing mutations or any other type of alteration located at the p17 sequence region upstream from position 1165.

It is an objective of the invention to provide primers which provides superior success rates for the amplification of the entire gag-protease genetic sequence from different HIV clades, i.e. clades with gag sequences carrying different additions, deletions or mutations, and protease sequences with diverse mutational profiles.

It is an objective of the invention to provide alternative primers which are able to amplify the entire gag-protease genetic sequence.

It is an objective of the invention to provide amplicons of the entire gag and protease genes which are useful for both genotyping and phenotyping experiments.

It is an objective of the invention to provide a suitable amplification method which is able to amplify in a reliable manner the entire gag and protease genes of any given sample.

It is an objective of the invention to provide amplicons of the entire gag and protease genes which are suitable in recombining with the target plasmid thereby generating infectious virus.

It is an objective of the invention to provide primers which provides superior success rates for the sequencing of the entire gag-protease genetic sequence from different HIV clades, i.e. clades with gag sequences carrying different additions, deletions or mutations, and protease sequences with diverse mutational profiles.

It is an objective of the invention to provide alternative primers which are able to sequence the entire gag-protease genetic sequence.

It is an objective of the invention to provide a method able to genotype the entire gag and protease genes from different HIV clades, i.e. clades with gag sequences carrying different additions, deletions or mutations, and protease sequences with diverse mutational profiles.

It is an objective of the invention to provide an alternative genotyping method of the entire gag and protease genes.

It is an objective of the invention to provide a method able to phenotype the entire gag and protease genes from different HIV clades, i.e. clades with gag sequences carrying different additions, deletions or mutations, and protease sequences with diverse mutational profiles.

It is an objective of the invention to provide an alternative phenotyping method of the entire gag and protease genes.

It is an objective of the invention to provide suitable vectors carrying a deletion of the entire gag and protease genes which are sufficiently stable, and are suitably designed for an optimal recombination with the target gag-protease amplicon.

It is an objective of the invention to provide a suitable recombination method which has an improved rate of success in generating infectious virus.

It is an objective of the invention to provide methods which are able to phenotype the entire gag and protease genes and which can mimic an in vivo setting.

It is an objective of the invention to provide a genotyping and a phenotyping method which enable to correlate the alterations found in the gag and protease genes, and resistance profiles.

It is a general objective of the invention to provide methods which are at least one of the following: simple, shorter, flexible, requiring less testing steps, requiring minimal manual intervention.

It is a general objective of the invention to provide methods which are able to genotype or phenotype the entire gag and protease genes of HIV-1 particles isolated either from patient plasma or culture supernatant harvested during resistance experiments.

It is a general objective of the invention to provide standardized methods which are able to genotype or phenotype the entire gag and protease genes.

The present invention meets one or more of these objectives by providing means and methods which successfully generate an amplicon which is useful for both genotyping and phenotyping the entire gag and protease genes.

As such, the present invention provides the means and methods for phenotypic and genotypic evaluation of the drug efficacy of gag and protease inhibitors based on the analysis of viral strains. Assays for evaluating the wild-type (WT) or mutant HIV gag and protease genetic sequences are disclosed, using a set of primers designed for the retrieval, preparation and analysis of HIV genetic material, and using a set of vectors suitable for creating recombinant virus encompassing the wild-type (WT) or mutant HIV gag and protease genetic sequences to be evaluated.

Thus, the present invention relates to a primer selected from SEQ ID no. 1-15.

The primer SEQ ID. No. 1 is useful for the reverse transcription of a HIV RNA sequence to obtain an HIV DNA sequence comprising the gag and protease genetic sequences or a portion thereof.

The primers SEQ ID. No. 2-4 are useful for the amplification of a HIV DNA sequence to obtain an amplicon comprising the gag and protease genetic sequences or a portion thereof.

TABLE 1

Primers for the reverse transcription of a HIV
RNA and amplification of a HIV DNA sequence
to obtain an amplicon comprising the gag and
protease genetic sequences or a portion thereof.

| SEQ | | | |
|---|---|---|---|
| ID. | | | |
| NO. | | SENSE | SEQUENCE |
| 1 | RET | reverse | 5′-ccattgtttaacttttgggccatcc-3″ |
| 2 | OUTER | forward | 5′-caagtagtgtgtgcccgtctgt-3′ |
| 3 | INNER | forward | 5′-tggaaaatctctagcagtggcg-3′ |
| 4 | | reverse | 5′-ccattcctggctttaattttactgg-3′ |

The invention further relates to the vectors or plasmids, described in the experimental part and the sequence listing, and to the use of these vectors in the methods disclosed herein. Both terms vector and plasmid are used in an equivalent meaning herein.

The vectors of the invention comprise the HIV genome and a deletion of the entire gag and protease genes. In a particular embodiment, the vectors encompass a deletion of the entire gag and protease genes, starting from the 49th base before the gag gene and ending at the 11th base after the protease gene.

In an embodiment, the invention provides the plasmids pUC19-5′HXB2d_MunI (SEQ ID no. 16), pUC19-5′HXB2d-delGP (SEQ ID no. 17), and pGEM-HIVdelGP (SEQ ID no. 18).

The plasmid pUC19-5′HXB2d_MunI (SEQ ID no. 16) may be prepared by creating a mutation for the insertion of a restriction site at the 4^{th} aminoacid of the 5′ RT gene on the starting plasmid pUC19-5′HXB2d. This starting material, i.e. plasmid pUC19-5′HXB2d (SEQ ID no. 19), contains the 5′ end of the HXBII virus from LTR to VPR and was constructed by digestion of pHXB2d with XbaI after which this fragment was digested with SalI. The resulting 6.8 kb fragment, being the 5′ end of the virus was then subcloned into pUC19.

The plasmid pUC19-5′HXB2d-delGP (SEQ ID no. 17) may be prepared by digesting the plasmid pUC19-5′HXB2d_MunI (SEQ ID no. 16), obtained in the method of the previous paragraph, with the enzymes BssHII and MfeI, and closing by ligation using a linker with a unique BstEII site.

The plasmid pGEM-HIVdelGP (SEQ ID no. 18) may be prepared by digesting the pUC19-5′HXB2d-delGP (SEQ ID no. 17) obtained in the method of the previous paragraph, with SfiI and XcmI and selecting the material with the biggest band; digesting of pGEM_HIVdelGPRT with SfiI and XcmI and selecting the material with the biggest band; and finally ligating the 2 materials having the biggest bands obtained in the previous steps. The starting material, i.e. plasmid pGEM_HIVdelGPRT, which has the accession number LMBP-4568, and is described in EP1283272.

A suitable plasmid backbone for the generation of the plasmids of the present invention may be selected from the group including, but not being limited to, pUC, pBR322 and pGEM.

The present invention further relates to the plasmids or vectors obtainable by the methods described herein.

In a further embodiment, the invention relates to the use of the plasmids or vectors obtainable by the methods described herein, for the preparation of a recombinant virus.

In one embodiment the invention provides a method for amplifying the gag and protease genetic sequences of a human immunodeficiency virus (HIV) comprising:
i) extracting HIV RNA or DNA sequences from a sample, wherein the HIV RNA or DNA sequences comprise at least a gag and a protease genetic sequence or a portion thereof;
i.a) optionally reverse-transcribing the HIV RNA sequence to obtain an HIV DNA sequence comprising the gag and protease genetic sequences or a portion thereof;
ii) amplifying the HIV DNA sequence to obtain an amplicon comprising the gag and protease genetic sequences or a portion thereof;
   characterized in that the optional reverse-transcription of the HIV RNA of step i.a) is performed with primer SEQ ID no. 1; and the amplification of the HIV DNA sequence of step ii) is performed with primers SEQ ID. No. 2-4; and provided that any one of the steps of the method for amplifying the gag and protease genetic sequences is not practised on the human or animal body.

In a further embodiment, the invention provides a method for determining the nucleotide sequence of the gag and protease genes of a human immunodeficiency virus (HIV) comprising the sequencing of the amplicon as obtained in step ii) in the method of the previous paragraph, using at least 8 of the primers selected from SEQ ID. no. 5-15. Alternatively, the method for determining the nucleotide sequence of the gag and protease genes of a human immunodeficiency virus (HIV) may also be performed by sequencing a plasmid containing the amplicon, as obtained in step ii) in the method of the previous paragraph, using at least 8 of the primers selected from SEQ ID. no. 5-15. The amplicon is inserted into the plasmid by sub-cloning procedures generally known by the skilled in the art.

The invention also relates to the nucleotide sequence of the gag and protease genes of a human immunodeficiency virus (HIV) determined by the method according to the previous paragraph.

The primers SEQ ID. No. 5-15 are useful for the sequencing of the amplicon comprising the gag and protease genetic sequences or a portion thereof At least 8 of the primers may be selected from SEQ ID. No. 5-15 for the sequencing of the amplicon comprising the gag and protease genetic sequences or a portion thereof These particular selections have the advantage that it enables the sequencing of the complete HIV gag and protease genes. Consequently, using these sets of primers all possible mutations that may occur in the HIV gag and protease genes may be resolved.

TABLE 2

Primers for the sequencing of the amplicon
comprising the gag and protease genetic
sequences or a portion thereof

| | | |
|---|---|---|
| SEQ ID. NO. | SENSE | SEQUENCE |

| | | |
|---|---|---|
| 5 | Forward | 5′-tttgactagcggaggctagaag-3′ |
| 6 | Forward | 5′-gacaagatagaggaagagca-3′ |
| 7 | Forward | 5′-catagcaggaactactagta-3′ |
| 8 | Forward | 5′-atgacagcatgtcagggagt-3′ |
| 9 | Forward | 5′-attatcagaaggagccac-3′ |
| 10 | Forward | 5′-aagacaccaaggaagc-3′ |
| 11 | Reverse | 5′-tctacatagtctctaaaggg-3′ |
| 12 | Reverse | 5′-gtggggctgttggctctggt-3′ |
| 13 | Reverse | 5′-tcttgtggggtggctccttc-3′ |
| 14 | Reverse | 5′-gataaaacctccaattcc-3′ |
| 15 | Reverse | 5′-ttatccatcttttat-3′ |

The genotype of the patient-derived gag and protease coding region may be determined directly from the amplified DNA by performing DNA sequencing after the amplification step. A variety of commercial sequencing enzymes and equipment may be used in this process. The efficiency may be increased by determining the sequence of the gag and protease coding regions in several parallel reactions, each with a different set of primers. Such a process could be performed at high throughput on a multiple-well plate, for example. Commercially available detection and analysis systems may be used to determine and store the sequence information for later analysis. The nucleotide sequence may be obtained using several approaches including sequencing nucleic acids. This sequencing may be performed using techniques including gel based approaches, mass spectroscopy and hybridization. However, as more resistance related mutations are identified, the sequence at particular nucleic acids, codons, or short sequences may be obtained. If a particular resistance associated mutation is known, the nucleotide sequence may be determined using hybridization assays (including Biochips, LipA-assay), mass spectroscopy, allele specific PCR, or using probes or primers discriminating between mutant and wild-type sequence. For these purposes the probes or primers may be suitably labeled for detection (e.g. Molecular beacons, TaqMan®, SunRise primers).

In one embodiment, the invention further provides a method for the preparation of a recombinant virus, said method comprising the homologous recombination of the amplicon comprising the gag and protease genetic sequences or a portion thereof, as obtained from the amplification of the HIV DNA sequence, with a vector encompassing a deletion of the gag and protease regions. In a particular embodiment, the homologous recombination is performed with one of the vectors described herein.

The present invention further relates to the recombinant virus obtainable by the methods described herein.

The invention further relates to a method for determining the phenotypic susceptibility of a human immunodeficiency virus to at least one drug, comprising the monitoring of the replicative capacity of the recombinant virus obtainable by the methods described herein in the presence of at least one cell and the at least one drug.

The replication capacity is the percentage of virus replication relative to the reference virus strain, e.g. NL4-3.

In one embodiment, the replicative capacity of the recombinant virus is compared to the replicative capacity of an HIV virus with mutant gag and protease genetic sequences in the presence of the same at least one drug.

The methods for determining the phenotypic susceptibility may be useful for designing a treatment regimen for an HIV infected patient, wherein the treatment regimen is selected based on the phenotypic susceptibility determined according to the methods described herein, and wherein the amplicon, which is obtained according to the methods described herein and which is recombined with a vector according the invention, it is obtained from the HIV RNA or DNA sequences extracted from a sample of the HIV infected patient.

For example, a method may comprise determining the relative replicative capacity of a clinical isolate of a patient and using said relative replicative capacity to determine an appropriate drug regimen for the patient.

The present invention also provides a method of identifying a drug effective against the HIV gag and/or protease genes comprising the production of an amplicon comprising the gag and protease genetic sequences or a portion thereof, determining the phenotypic susceptibility of this amplicon—in a recombined form—towards said drug, and using said phenotypic susceptibility results to determine the effectiveness of said drug.

The invention further relates to a method for determining the genotypic alterations in the HIV gag and protease nucleotide sequences comprising the comparison of the nucleotide sequence as determined by the methods described herein, with the gag and protease nucleotide sequences of a wild-type HIV virus.

A viral sequence may contain one or multiple alterations in the gag and protease genetic sequences when compared to the consensus wild-type sequence. A single alteration or a combination thereof may correlate to a change in drug efficacy. This correlation may be indicative of reduced or increased susceptibility of the virus towards a drug. Said alterations may also influence the viral fitness. Alterations in the patient borne HIV gag and protease genetic sequences may be identified by sequence comparison with a reference sequence of a viral strain e.g. K03455. K03455 is present in Genbank and available through the internet. The identified alterations may be indicative of a change in susceptibility of the viral strain for one or more drugs. Said susceptibility data are derived from phenotypic analysis, wherein the gag and protease sequences comprising said alterations are analyzed.

The present invention further provides a genotypic and phenotypic database of HIV gag and protease sequences or portions thereof, comprising:
a nucleotide sequence as determined by the methods described herein;
a nucleotide sequence of the gag and protease genes of a wild-type HIV;
the phenotypic susceptibility of the nucleotide sequence as obtained by the methods described herein, said phenotypic susceptibility being determined according to the methods described herein;
the phenotypic susceptibility of the nucleotide sequence of the gag and protease genes of a wild-type HIV, which is determined by the methods described herein;
a data table with the correlation of each of the nucleotide sequences with their corresponding phenotypic susceptibility.

The present invention further provides a method for determining the phenotypic susceptibility to at least one drug of the nucleotide sequence of the gag and protease genes as determined by the methods described herein, comprising the querying of the data table described above to obtain the maximum concordance with the nucleotide sequence; wherein the degree of concordance is indicative of the phenotypic susceptibility. Basically, the method allows the comparison of a given gag and protease sequence with sequences present in a database, of which the phenotypic susceptibility has been determined with the methods of the present invention, and using said sequence comparison to determine the effectiveness of said drug.

Results from phenotyping and genotyping experiments can be used to develop a database of replicative capacity levels in the presence of particular drugs, drug regimens or other treatment for a large number of mutant HIV strains. One such approach is virtual phenotyping (WO 01/79540). Briefly, the genotype of a patient derived gag and protease sequences may be correlated to the phenotypic susceptibility of said patient derived gag and protease sequences. In an alternative operation, if no phenotyping is performed, the test gag and protease sequence may be screened towards a collection of sequences present in a database. Identical sequences are retrieved and the database is further interrogated to identify if a corresponding phenotype is known for any of the retrieved sequences. In this latter case a virtual phenotype may be determined (see also infra). A report may be prepared including the susceptibility of the viral strain for one or more therapies, the sequence of the strain under investigation, biological cut-offs. Suitably, complete sequences will be interrogated in the database. Optionally, portions of sequences, such as combinations of mutations or alterations indicative of a change in drug susceptibility, may as well be screened. Such combination of mutations is sometimes referred to as a hot-spot (see e.g. WO 01/79540). Additionally, data may then be incorporated into existing programs that analyze the drug susceptibility of viruses with mutations in other segments of the HIV genome such as in the pol genes. For example, such a database may be analyzed in combination with reverse transcriptase sequence information and the results used in the determination of appropriate treatment strategies.

Thus, obtained phenotypic and genotypic data enable the development of a database comprising both phenotypic and genotypic information of the HIV gag and protease genes, wherein the database further provides a correlation in between genotypes and genotypes, and in between genotypes and phenotypes, wherein the correlation is indicative of efficacy of a given treatment regimen. Such a database can further be used to predict the phenotype of the HIV gag and proteases gene based on its genotypic profile.

In addition, the present invention relates as well to kits useful for amplifying and sequencing the HIV gag and protease genetic sequences; thereby allowing the phenotyping and genotyping of the HIV gag and protease genes. Such kits for determining the susceptibility of at least one HIV virus to at least one drug may comprise the primers SEQ ID No. 1-4. In another embodiment, the kits mentioned above may further comprise a plasmid as described in the present invention. For the purpose of performing the phenotyping assay, such kits may be further completed with at least one HIV inhibitor. Optionally, a reference plasmid bearing a wild type HIV sequence may be added. Optionally, cells susceptible of HIV transfection may be added to the kit. In addition, at least one reagent for monitoring the replicative capacity of recombinant virus may be added. In a particular embodiment, this at least one reagent is an indicator gene or reporter molecule such as enzyme substrates.

The present invention also relates to a kit for genotyping the HIV gag and protease genes. Such kit comprises at least one of the primers selected from SEQ ID No. 1-15. Optionally, additional reagents for performing the nucleic amplification and subsequent sequence analysis may be added. Reagents for cycle sequencing may be included. The primers may be fluorescently labeled.

Optionally, a full kit for genotyping and phenotyping the HIV gag and protease genes may be assembled.

A human immunodeficiency virus (HIV), as used herein refers to any HIV including laboratory strains, wild type strains, mutant strains and any biological sample comprising at least one HIV virus, such as, for example, an HIV clinical isolate. HIV strains compatible with the present invention are any such strains that are capable of infecting mammals, particularly humans. Examples are HIV-1 and HIV-2. For reduction to practice of the present invention, an HIV virus refers to any sample comprising at least one HIV virus. Since a patient may have HIV viruses in his body with different alterations in the gag and protease genes, it is to be understood that a sample may contain a variety of different HIV viruses containing different alterations or mutational profiles in the gag and protease genes. A sample may be obtained for example from an individual, from cell cultures, or generated using recombinant technology, or cloning. HIV strains compatible with the present invention are any such strains that are capable of infecting mammals, particularly humans.

Viral strains used for obtaining a plasmid are preferably HIV wild-type sequences, such as LAI, HXB2D. LAI, also known as IIIB, is a wild-type HIV strain. One particular clone thereof, this means one sequence, is HXB2D. This sequence may be incorporated into a plasmid.

Instead of viral RNA, HIV DNA, e.g. proviral DNA, may be used for the methods described herein. In case RNA is used, reverse transcription into DNA by a suitable reverse transcriptase is needed. The protocols describing the analysis of RNA are also amenable for DNA analysis. However, if a protocol starts from DNA, the person skilled in the art will know that no reverse transcription is needed. The primers designed to amplify the RNA strand, also anneal to, and amplify DNA. Reverse transcription and amplification may be performed with a single set of primers. Suitably a hemi-nested and more suitably a nested approach may also be used to reverse transcribe and amplify the genetic material.

Nucleic acid may be amplified by techniques such as polymerase chain reaction (PCR), nucleic acid sequence based amplification (NASBA), self-sustained sequence replication (3SR), transcription-based amplification (TAS), ligation chain reaction (LCR). Often PCR is used.

For the purpose of the present invention an amplicon refers to the amplified and, where necessary, reverse-transcribed gag and protease genetic sequences or portions thereof It should be understood that these gag and protease genetic sequences may be of diverse origin, including plasmids and patient material; suitably it is obtained from patient derived material. Amplicon is sometimes defined as the “DNA construct”.

A portion of the gag and protease genes is defined as a fragment of the gag and protease genes recovered from patient borne virus, lab viruses including IIIB and NL4-3, or mutant viruses. This fragment does not encompass the complete gag and protease genes. Said fragment may be obtained directly from its source, including a patient sample, or may be obtained using molecular biology tools following the recovery of the complete gag and protease sequences.

Primers specific for the gag and protease regions of the HIV genome such as the primers described herein and their homologues are claimed. Such primers are chosen from SEQ. ID N^{o} 1-15 or have at least 80% homology, preferably 90% homology, more preferably 95% homology as determined using algorithms known to the person skilled in the art such as FASTA and BLAST. Interesting sets of primers include at least one primer selected from SEQ. ID N^{o} 1, SEQ. ID N^{o} 2-4, SEQ. ID N^{o} 5-15, and SEQ. ID N^{o} 5, 7-8, 10-14. The primer sequences listed herein may be labeled.

Suitably, this label may be detected using fluorescence, luminescence or absorbance. In addition primers located in a region of 50 nucleotides (nt) upstream or downstream from the sequences given herein constitute part of the present invention. Specifically, the primers may be located in a region of 20 nt upstream or downstream from the sequences given herein and, constitute, as well, part of the present invention. Also, primers comprising at least 8 consecutive bases present in either of the primers described herein constitute an embodiment of the invention. Interestingly, the primers comprise at least 12 consecutive bases present in either of the primers described herein. In one aspect of the present invention the primers may contain linker regions for cloning. Optionally, the linker region of a primer may contain a restriction enzyme recognition site. Preferably, said restriction enzyme recognition site is a unique restriction enzyme recognition site. Alternatively, primers may partially anneal to the target region.

A drug means any agent such as a chemotherapeutic, peptide, antibody, antisense, ribozyme and any combination thereof be it marketed or under development. Examples of drugs include those compounds having antiretroviral activity such as suramine, pentamidine, thymopentin, castanospermine, dextran (dextran sulfate), foscarnet-sodium (trisodium phosphono formate); nucleoside reverse transcriptase inhibitors (NRTIs), e.g. zidovudine (AZT), didanosine (ddI), zalcitabine (ddC), lamivudine (3TC), stavudine (d4T), emtricitabine (FTC), abacavir (ABC), D-D4FC (Reverset™), alovudine (MIV-310), amdoxovir (DAPD), elvucitabine (ACH-126,443), and the like; non-nucleoside reverse transcriptase inhibitors (NNRTIs) such as delarvidine (DLV), efavirenz (EFV), nevirapine (NVP), capravirine (CPV), calanolide A, TMC120, etravirine (TMC125), TMC278, BMS-561390, DPC-083 and the like; nucleotide reverse transcriptase inhibitors (NtRTIs), e.g. tenofovir (TDF) and tenofovir disoproxil fumarate, and the like; compounds of the TIBO (tetrahydroimidazo-[4,5,1-jk][1,4]-benzodiazepine-2(1H)-one and thione)-type e.g. (S)-8-chloro-4,5,6,7-tetrahydro-5-methyl-6-(3-methyl-2-butenyl)imidazo-[4,5,1-jk][1,4]-benzodiazepine-2(1H)-thione; compounds of the α-APA (α-anilino phenyl acetamide) type e.g. α-[(2-nitrophenyl)amino]-2,6-dichlorobenzene-acetamide and the like; inhibitors of trans-activating proteins, such as TAT-inhibitors, e.g. RO-5-3335; REV inhibitors; protease inhibitors e.g. ritonavir (RTV), saquinavir (SQV), lopinavir (ABT-378 or LPV), indinavir (IDV), amprenavir (VX-478), TMC-126, BMS-232632, VX-175, DMP-323, DMP-450 (Mozenavir), nelfinavir (AG-1343), atazanavir (BMS 232,632), palinavir, TMC-114, R0033-4649, fosamprenavir (GW433908 or VX-175), P-1946, BMS 186,318, SC-55389a, L-756,423, tipranavir (PNU-140690), BILA 1096 BS, U-140690, and the like; entry inhibitors which comprise fusion inhibitors (e.g.

T-20, T-1249), attachment inhibitors and co-receptor inhibitors; the latter comprise the CCRS antagonists and CXR4 antagonists (e.g. AMD-3100); examples of entry inhibitors are enfuvirtide (ENF), GSK-873,140, PRO-542, SCH-417,690, TNX-355, maraviroc (UK-427,857); gag processing inhibitors (or maturation inhibitors) such as PA-457 (Panacos Pharmaceuticals); inhibitors of the viral integrase; ribonucleotide reductase inhibitors (cellular inhibitors), e.g. hydroxyurea and the like; capsid protein polymerization inhibitors; budding inhibitors or assembly inhibitors.

In particular, agents interfering with HIV gag and protease biology are analyzed.

Treatment or treatment regimen refers to the management or handling of an individual medical condition by the administration of drugs, at directed dosages, time intervals, duration, alone or in different combinations, via different administration routes, in suitable formulations, etc.

The susceptibility of at least one HIV virus to at least one drug is determined by the replicative capacity of the recombinant virus in the presence of at least one drug relative to the replicative capacity of an HIV virus with wild-type gag and protease genetic sequences in the presence of the same at least one drug. Replicative capacity means the ability of the virus or chimeric construct to grow under culturing conditions. This is sometimes referred to as viral fitness. The culturing conditions may contain triggers that influence the growth of the virus, examples of which are drugs.

An alteration in viral drug sensitivity is defined as a change in susceptibility of a viral strain to said drug. Susceptibilities are generally expressed as ratios of EC₅₀ or EC₉₀ values. The EC₅₀ or EC₉₀ value is the effective drug concentration at which 50% or 90% respectively of the viral population is inhibited from replicating. The IC₅₀ or IC₉₀ value is the drug concentration at which 50% or 90% respectively of the enzyme activity is inhibited. Hence, the susceptibility of a viral strain can be expressed as a fold change in susceptibility, wherein the fold change is derived from the ratio of, for instance, the EC₅₀ or IC₅₀ values of a mutant viral strain, compared to the wild type EC₅₀ or IC₅₀ values. In particular, the susceptibility of a viral strain or population may also be expressed as resistance of a viral strain, wherein the result is indicated as a fold increase in EC₅₀ or IC₅₀ as compared to wild type EC₅₀ or IC₅₀.

The susceptibility of at least one HIV virus to one drug may be tested by determining the cytopathogenicity of the recombinant virus to cells. In the context of this invention, the cytopathogenic effect means, the viability of the cells in culture in the presence of chimeric viruses.

The cells may be chosen from T cells, monocytes, macrophages, dendritic cells, Langerhans cells, hematopoietic stem cells or, precursor cells, MT4 cells and PM-1 cells. Suitable host cells for homologous recombination of HIV sequences include MT4 and PM-1. MT4 is a CD4⁺T-cell line containing the CXCR4 co-receptor. The PM-1 cell line expresses both the CXCR4 and CCRS co-receptors. All the above mentioned cells are capable of producing new infectious virus particles upon recombination of the gag/protease deletion vectors with the gag/protease amplicons. Thus, they can also be used for testing the cytopathogenic effects of recombinant viruses.

The cytopathogenicity may, for example, be monitored by the presence of a reporter molecule, including reporter genes. A reporter gene is defined as a gene whose product has reporting capabilities. Suitable reporter molecules include tetrazolium salts, green fluorescent proteins, beta-galactosidase, chloramfenicol transferase, alkaline phosphatase, and luciferase. Several methods of cytopathogenic testing including phenotypic testing are described in the literature comprising the recombinant virus assay (Kellam and Larder, Antimicrob. Agents Chemotherap. 1994, 38, 23-30, Hertogs et al. Antimicrob. Agents Chemotherap. 1998, 42, 269-276; Pauwels et al. J. Virol Methods 1988, 20, 309-321).

The term chimeric means a construct comprising nucleic acid material from different origin such as, for example, a combination of wild type virus with a laboratory virus, a combination of wild type sequence and patient derived sequence.

The amplicons or sequences to be specifically detected by sequence analysis and to be recombined into infectious virus according to the present invention may be wild type, polymorphic or mutant sequences of the HIV gag and protease genes or fragments thereof These amplicons or sequences may encompass one or several nucleotide changes. In the present invention said amplicons or sequences often include one or two variable nucleotide positions. Sequence alterations detected and analysed by the methods of the invention include, but are not limited to, single nucleotide mutations, substitutions, deletions, insertions, transversions, inversions, repeats or variations covering multiple variations, optionally present at different locations. Sequence alterations may further relate to epigenetic sequence variations not limited to for instance methylation.

Any type of patient sample may be used to obtain the gag and protease genes, such as, for example, serum and tissue. Viral RNA may be isolated using known methods such as that described in Boom, R. et al. (J. Clin. Microbiol. 28(3): 495-503 (1990)). Alternatively, a number of commercial methods such as the QIAAMP® viral RNA kit (Qiagen, Inc.) may be used to obtain viral RNA from bodily fluids such as plasma, serum, or cell-free fluids. DNA may be extracted from tissue using methods known by the skilled in the art such as the procedure described by Maniatis et al. (1982) which involves the preparation of a cell lysate followed by digestion with proteinase K, obtaining DNA purification by a multi-step phenol extraction, ethanol precipitation and ribonuclease digestion. Optionally, available commercial methods may also be employed to obtain DNA from bodily fluids, such as QIAAMP® Blood kits for DNA isolation from blood and body fluids (Qiagen, Inc.)

To prepare recombinant HIV viruses for phenotyping assays, the amplified sequences of the gag and protease genes, or portions thereof, also termed herein as amplicons, may be inserted into a vector comprising the wild-type HIV sequence with a deletion of the relevant portion. An infectious clone is generated upon exchange of genetic material between the amplicon and the deletion construct to yield an HIV sequence.

In the present invention, there is further disclosed a method for obtaining a plasmid containing the wild-type HIV sequence with a deletion in the gag and protease regions of the HIV genome. The removal of the region of interest is achieved by amplification of a plasmid containing the wild type HIV sequence, such as HXB2D. This plasmid amplification refers to the selective amplification of a portion of the plasmid using primers annealing to the flanking sequences of the desired deletion region, i.e. gag and protease sequences, such that all plasmid is amplified except for the region to be deleted. Such method of amplification is a direct, one-step, and simple technique to produce a deletion of a sequence in a circular plasmid DNA.

Preferably, said amplification of plasmid's DNA generates a unique restriction site. Unique restriction site refers to a single occurrence of a site on the nucleic acid that is recognized by a restriction enzyme or that it does not occur anywhere else in the construct. The unique restriction site may be created after amplification by re-ligating both 5′ and 3′ ends of the amplified plasmid's DNA. Alternatively, the unique restriction site may not have to be created, as it may be already fully located in one of the ends, 5′ or 3′ end. Optionally, the unique restriction site may be inserted. Optionally, part of the unique restriction site may be present in the region to be amplified. The creation of a unique restriction site deriving from amplification is a preferred method since is a one-step, direct, simple and fast method. The unique restriction site is further relevant for the production of recombinant virus.

As one skilled in the art will understand, the creation of a unique restriction site will depend upon the sequence of the HIV genome, and upon the sequence to be deleted therefrom. Unique restriction sites that can be employed in the present invention are those present only once in the HIV genome and may flank the region of interest to be deleted. Optionally, the primers used for amplification may contain the same or other specific restriction endonuclease sites to facilitate insertion into a different vector. Additionally, one of the primers used for amplification may contain a phosphorylated 5′ end-linker to facilitate insertion of a foreign amplicon. One interesting unique restriction site is BstEII. Any other restriction sites not occurring in the HIV genome can be used to be inserted as a unique restriction site.

Optionally, the method for obtaining a plasmid containing the wild-type HIV sequence with a deletion in the gag and protease regions of the HIV genome, may be performed in a second cloning vector. The gag and protease regions may be inserted into a cloning vector such as pGEM (Promega, for example the backbone of pGEM3 vector has been used) and manipulated, by amplification, to remove part of the gag- and protease-coding regions such that insertion of the remaining gag-protease sequence from the samples would not disrupt the reading frame. The manipulated gag-protease region may then be placed in a pSV40HXB2D or a pSV62HXB2D vector such that it contains the complete wild type HIV sequence except for the relevant gag and protease deletions.

Examples of gag-protease deletion vectors are pUC19-5′HXB2d_MunI (SEQ ID no. 16), pUC19-5′HXB2d-delGP (SEQ ID no. 17), and pGEM-HIVdelGP (SEQ ID no. 18).

Those of skill in the art will appreciate that other HIV vectors and cloning procedures known in the art may also be used to create Agag-protease plasmids for recombination or ligation with patient derived sequences and creation of infectious viruses. For instance, deletion constructs may be prepared by re-introducing portions of the gag and protease genes into a plasmid wherein the gag and protease genes have been previously deleted by amplification. In general, vectors must be created to allow re-insertion of the deleted sequences without disrupting the reading frame of the gag and protease genes.

The amplified gag and protease sequences may be inserted into one of the Δgag-protease vectors by homologous recombination in a suitable host cell between overlapping DNA segments in the vector and amplified sequence. Alternatively, the amplified gag and protease sequences can be incorporated into the vector at a unique restriction site according to cloning procedures standard in the art. This latter is a direct cloning strategy. Suitable for direct cloning strategies is the use of two different restriction sites to facilitate ligation of the amplified region in the appropriate orientation.

It is convenient to insert both gag and protease sections into the vector even when mutations are only expected in one of these two sections. Recombinant viruses incorporating all of the gag and protease sequences would prevent incompatibility between mixed gag and protease subunits in a recombinant vector. Recombinant vectors bearing gag and protease subunits i.e. gag or protease or parts thereof from different origin, could yield incompatibility upon transfecting cell lines. Recombinant virus stocks may be stored for future analysis, such as for example, viability testing.

Following the generation of the recombinant construct the chimeric virus may be grown and the viral titer determined before proceeding to the determination of the phenotypic susceptibility. The titer of a viral population indicates the strength or potency of said viral population in infecting cells. The titer of a specific viral population can be defined as the highest dilution of said viral population giving a cytopathogenic effect (CPE) in 50% of inoculated cell cultures. The indicator gene, encoding a signal indicative of replication of the virus in the presence of a drug or indicative of the susceptibility of the virus in the presence of a drug may be present in the culturing cells such as MT-4 cells. Alternatively, said indicator gene may be incorporated in the chimeric construct introduced into the culturing cells or may be introduced separately. Suitable indicator genes encode for fluorescent proteins, particularly green fluorescent protein or mutant thereof In order to allow homologous recombination, genetic material may be introduced into the cells using a variety of techniques known in the art including, calcium phosphate precipitation, liposomes, viral infection, and electroporation. The monitoring may be performed in high throughput.

The protocols and products of the present invention may be used for diverse diagnostic, clinical, toxicological, research and forensic purposes including, drug discovery, designing patient therapy, drug efficacy testing, and patient management. The present methods may be used in combination with other assays. The results may be implemented in computer models and databases. The products described herein may be incorporated into kits.

Additionally, the protocols and products of the present invention also allow monitoring of the resistance profiles of anti-HIV compounds that target gag and protease gene products. They may also be useful in determining how the effectiveness of a variety of different types of anti-HIV compounds depends on gag-protease phenotype and genotype. For example, the assays of the present invention may be used for the detection of gag cleavage sites and the determination of the efficacy of anti-HIV compounds against PI-resistant HIV strains. Additionally, the activity of antivirals which target the gag and protease genes may be screened by running clinically significant HIV strains encompassing mutant gag and protease sequences, wild-type gag and protease sequences, or optionally in the presence of neutralizing antibodies, chemokines, or plasma proteins, in a phenotypic assay. In a similar embodiment, the phenotypic assay may be used as or comprise part of a high-throughput screening assay where numerous antivirals and HIV strains compositions are evaluated. The results may be monitored by several approaches including but not limited to morphology screening, microscopy, and optical methods, such as, for example, absorbance and fluorescence.

The assays of the present invention may as well be used for therapeutic drug monitoring. Said approach includes a combination of susceptibility testing, determination of drug level and assessment of a threshold. Said threshold may be derived from population based pharmacokinetic modeling (WO 02/23186). The threshold is a drug concentration needed to obtain a beneficial therapeutic effect in vivo. The in vivo drug level may be determined using techniques such as high performance liquid chromatography, liquid chromatography, mass spectroscopy or combinations thereof. The susceptibility of the virus may be derived from phenotyping or interpretation of genotyping results.

In addition, the assays of the present invention may also be useful to discriminate an effective drug from an ineffective drug by establishing cut-offs, i.e. biological cut-offs (see e.g. WO 02/33402). Biological cut-offs are drug specific. These cut-offs are determined following phenotyping of a large population of individuals mainly containing wild-type viruses. The cut-off is derived from the distribution of the fold increase in resistances of the wild-type viruses for a particular drug.

### BRIEF DESCRIPTION OF THE DRAWINGS

FIG. 1. Overview of the amplification primers and their location on the HIV viral genome. EF1 corresponds to primer with SEQ ID no. 2, gaprout-R3 corresponds to primer with SEQ ID no. 1, IF1 corresponds to primer with SEQ ID no. 3, and gaprout-R1 corresponds to primer with SEQ ID no. 4.

FIG. 2. Overview of the cloning strategy

FIG. 3: Schematic representation of the subcloning of deletion construct pUC19-5′HXB2d-delGP (SEQ ID no. 17)

FIG. 4. Schematic representation of the subcloning of deletion construct pGEM-HIVdelGP (SEQ ID no. 18)

FIG. 5. Schematic representation of the pUC19-5′HXB2d_MunI vector (SEQ ID no. 16)

FIG. 6. Schematic representation of the pUC19-5′HXB2d-delGP vector (SEQ ID no. 17)

FIG. 7. Schematic representation of the pGEM-HIVdelGP vector (SEQ ID no. 18)

FIG. 8. Schematic representation of the pUC19-5′HXB2d vector (SEQ ID no. 19)

FIG. 9: Graph showing the phenotype (expressed as Fold-change in susceptibility, i.e. FC) of 2 viruses (one original laboratory strain and one recombinant virus, having both virus the same gag and protease genes), when tested in the presence of the antiviral drugs: amprenavir (APV), atazanavir (ATV) and lopinavir (LPV).

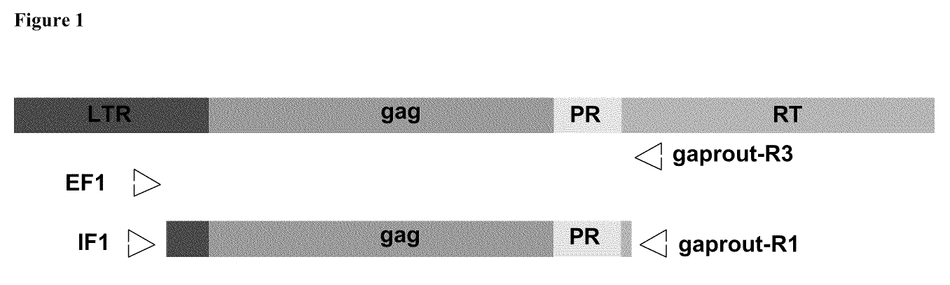

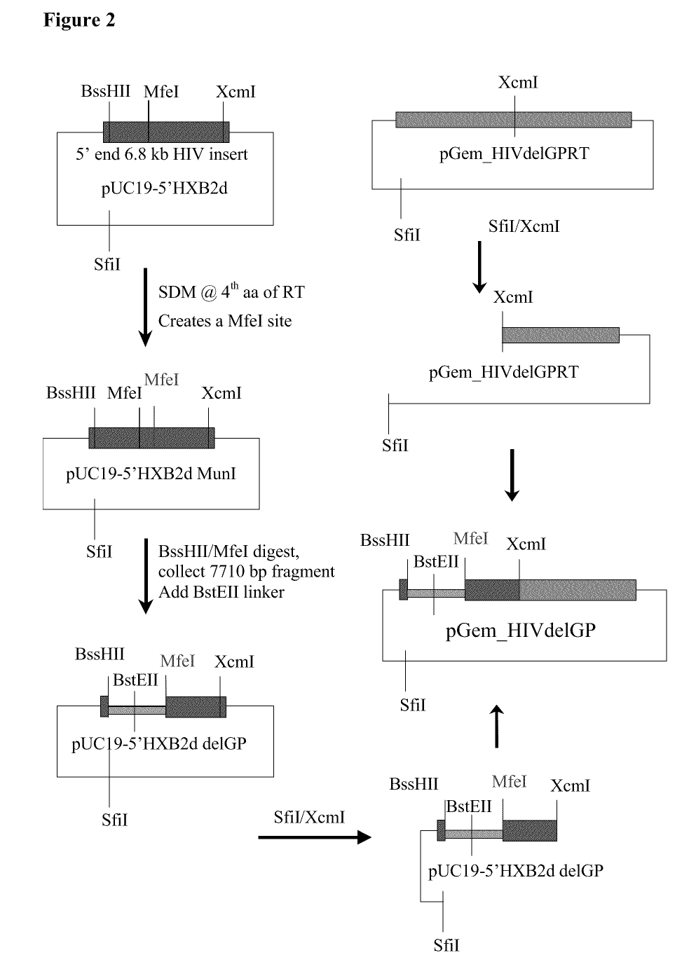

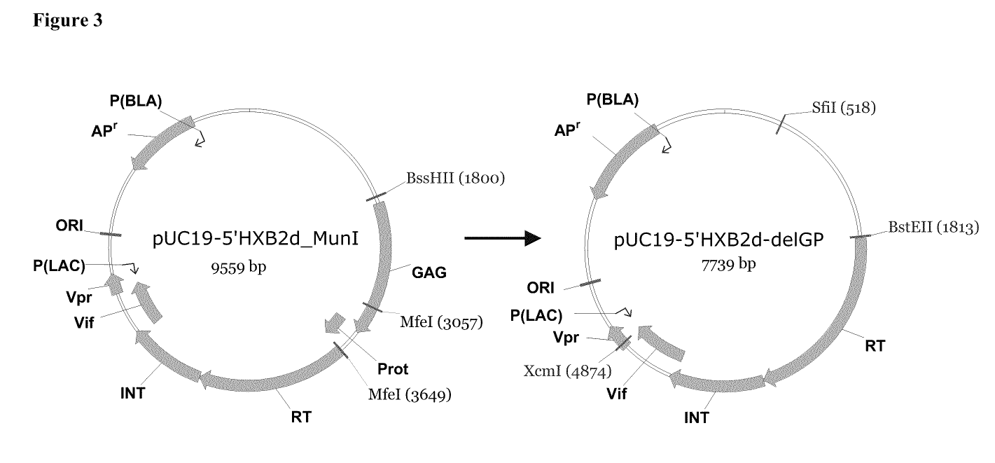

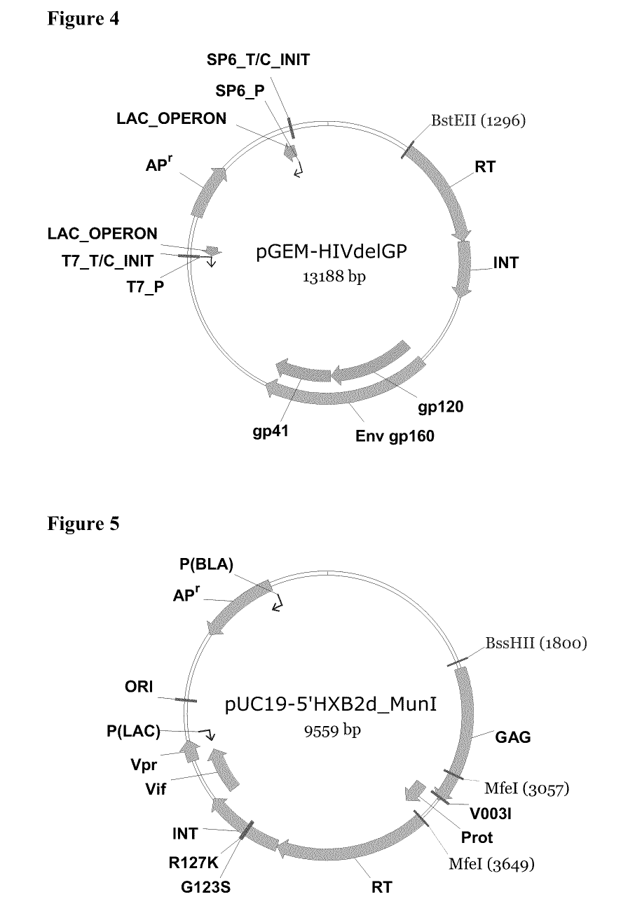

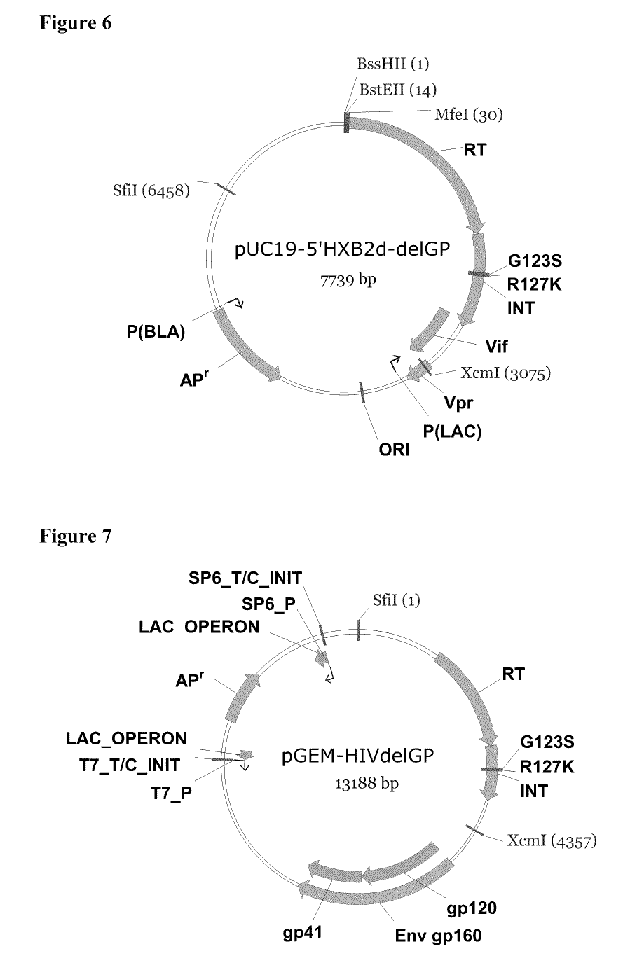

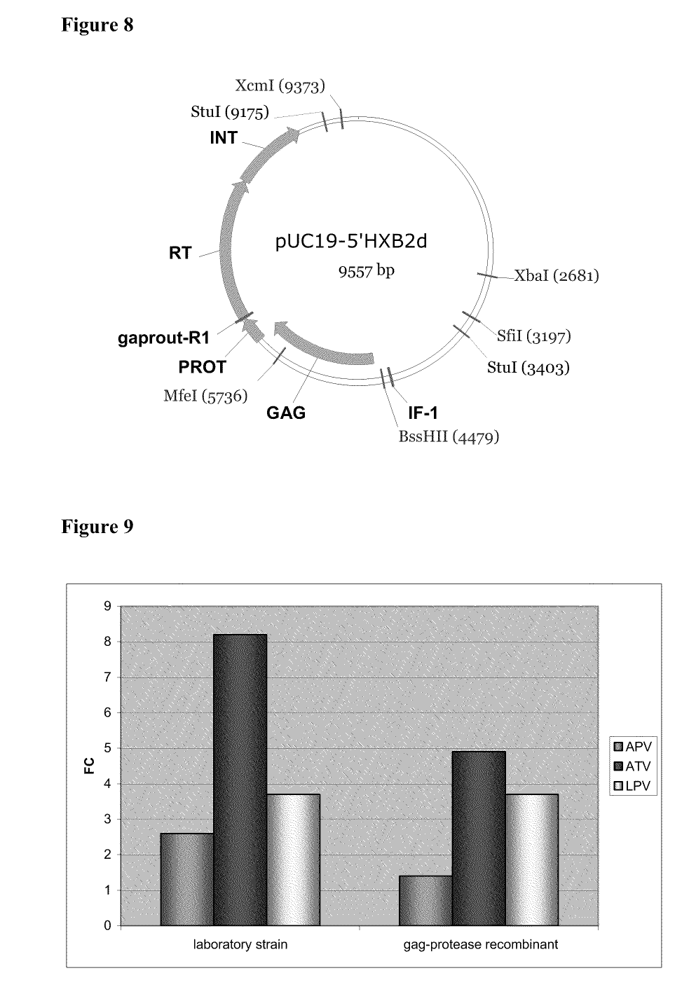

The following non-limiting examples help to illustrate the principles of the invention.

### EXPERIMENTAL PART

### Example 1

### Extraction and Amplification of Viral RNA

RNA was isolated from 100 μl of plasma with the Qiagen viral RNA extraction kit, and reverse transcribed with the Expand Reverse Transcriptase (Roche) as described by the manufacturer and using an HIV-1 specific downstream primer (Gaprout-R3: 5′-CCATTGTTTAACTTTTGGGCCATCC 3′; SEQ ID NO: 1). PCR on reverse transcribed RNA was performed with outer (5′-CAAGTAGTGTGTGCCCGTCTGT-3′) and inner primers (Gaprout-R1: 5′- CCATTCCTGGCTTTAATTTTACTGG-3′ and IF1: 5′-TGGAAAATCTCTAGCAGTGGCG-3′). After purification with the QiaQuick PCR purification kit, the isolated PCR product was ready for use in transfection reactions.

The table 3 below shows the success rate of the amplification protocol as described in the previous paragraph on samples from different clades. The success rates are calculated as the percentage of the number of samples that were successfully amplified from a total of samples tested.

TABLE 3

| | Total amount of | |
|---|---|---|
| Clade | tested samples | success rates (%) |
| A | 12 | 92 |
| CRF01_AE | 3 | 100 |
| CRF02_AG | 8 | 75 |
| B | 42 | 95 |
| C | 7 | 100 |
| D | 5 | 80 |
| total | 77 | 92 |

### Example 2

### Production and Isolation of Plasmid

Production of pGEM-HIVdelGP plasmid (SEQ ID no. 18) was performed in E. coli. Plasmid DNA was isolated from overnight cultures making use of Qiagen columns as described by the manufacturer. The yield of the isolated plasmid was determined spectrophotometrically by A260/280 measurement (optical density measurement at X=260 and 280 nm). About 250 μg of ultrapure plasmid DNA was obtained from 500 ml of bacterial culture.

The identity of the isolated plasmid was confirmed by restriction analysis.

Subsequently, the isolated plasmid DNA was linearised with BstEII and purified again by a classical ethanol precipitation.

### Example 3

### Transfection of Cells

MT4 cells were subcultured at a density of 250,000 cells/ml before transfection (exponential growth phase). Cells were pelleted and resuspended in solution V at a concentration of 2.5×10⁶ cells/ml. Nucleofection was performed with the amaxa system as described in WO02/00871, WO02/086129, WO02/086134. Cells were electroporated in the presence of 1 μg of linearised pGEM-HIVdelGP plasmid (SEQ ID no. 18) and approximately 10 μg of RT PCR product. Incubation was performed at 37° C. in a humidified atmosphere of 5% C0₂.

### Example 4

### Culture and Follow-Up of Transfected Cells

During 7 to 10 days following the transfection, cells were monitored for the appearance of cytopathogenic effect (CPE). In the absence thereof, cells were subcultured in different flasks. Subsequently, cell culture supernatants were used to create a stock of recombinant virus and stored in 1.5 ml aliquots at −70 ° C.

From a total of 30 starting samples, 29 gave viable recombinant virus, and out of these 29, 25 recombinant viruses were successfully phenotyped.

### Example 5

### Analysis of Recombinant Virus from Viral RNA

After titration of the viruses, the viral stocks were used for antiviral experiments in the presence of serial dilutions of different HIV inhibitors. Titers of the harvested supernatants were determined by limited serial dilution titration of virus in MT4 cells.

Titrated viruses were used in antiviral experiments. For this purpose, 384-well microtiter plates were filled with complete culture medium. Subsequently, stock solutions of compounds were added. HIV- and mock-infected cell samples were included for each drug (or drug combination).

Exponentially growing MT4 cells were then transferred to the microtiter plates at a density of 150,000 cells/ml. The cell cultures were then incubated at 37° C. in a humidified atmosphere of 5% C0₂. Three days after infection, the viability of the mock- and HIV-infected cells was examined by measuring the fluorescent signal from the infected cells.

FIG. 9 illustrates the phenotype (expressed as Fold-change in susceptibility, i.e. FC) of 2 viruses (one original laboratory strain and one recombinant virus, having both virus the same gag and protease genes), when tested in the presence of the antiviral drugs: amprenavir (APV), atazanavir (ATV) and lopinavir (LPV).

The results confirmed that the phenotype of the recombinant virus is similar to the phenotype of the corresponding laboratory strain, therefore proving that the phenotyping method of the invention was able to mimic an in vivo setting, was standardized, and was able to test HIV-1 particles of different origin.

### Example 6

### Genotyping of the Recombinant Virus

The PCR products obtained from the recombinant virus samples were genotyped by dideoxynucleotide-based sequence analysis. Samples were sequenced using the Big Dye terminator kit (Applied Biosystems) and resolved on an ABI 3730 DNA sequencer. The following primers were used:

| Forward primers | |
|---|---|
| SEQ ID no. 5, F0 gag: | |
| 5′-TTTGACTAGCGGAGGCTAGAAG-3′ | (761-782) |
| SEQ ID no. 7, F3 gag: | |
| 5′-CATAGCAGGAACTACTAGTA-3′ | (1494-1513) |
| SEQ ID no. 8, F5 gag: | |
| 5′-ATGACAGCATGTCAGGGAGT-3′ | (1828-1847) |
| SEQ ID no. 10, F10 gag: | |
| 5′-AAGACACCAAGGAAGC-3′ | (1073-1088) |
| Reverse primers | |
| SEQ ID no. 11, R3 gag: | |
| 5′-TCTACATAGTCTCTAAAGGG-3′ | (1682-1663) |
| SEQ ID no. 12, R7 gag: | |
| 5′-GTGGGGCTGTTGGCTCTGGT-3′ | (2164-2145) |
| SEQ ID no. 13, R8 gag: | |
| 5′-TCTTGTGGGGTGGCTCCTTC-3′ | (1337-1318) |
| SEQ ID no. 14, R8 (GPRT): | |
| 5′-GATAAAACCTCCAATTCC-3′ | (2414-2397) |

The table 4 below shows the success rate of the genotyping protocol as described in the previous paragraph on the amplicons that were amplified following the protocol of example 1. The success rates are calculated as the percentage of the number of samples that were successfully genotyped from a total of samples tested.

TABLE 4

| | Total amount of | |
|---|---|---|
| Clade | tested samples | success rates (%) |
| A | 12 | 92 |
| CRF01_AE | 3 | 100 |
| CRF02_AG | 8 | 75 |
| B | 46 | 87 |
| C | 7 | 100 |
| D | 5 | 80 |

1. A primer selected from SEQ ID no. 1-15.

2. A vector comprising the HIV genome and a deletion of the entire gag and protease genes.

3. The vector according to claim 2 wherein the deletion of the entire gag and protease genes starts from the 49th base before the gag gene and ends at the 11th base after the protease gene.

4. A vector according to claim 2 wherein the vector is selected from pUC19-5′HXB2d_MunI (SEQ ID no. 16), pUC19-5′HXB2d-delGP (SEQ ID no. 17), and pGEM-HIVdelGP (SEQ ID no. 18),

5. A method for amplifying the gag and protease genetic sequences of a human immunodeficiency virus (HIV) comprising:
i) extracting HIV RNA or DNA sequences from a sample, wherein the HIV RNA or DNA sequences comprise at least a gag and a protease genetic sequence or a portion thereof;
i.a) optionally reverse-transcribing the HIV RNA sequence to obtain an HIV DNA sequence comprising the gag and protease genetic sequences or a portion thereof;
ii) amplifying the HIV DNA sequence to obtain an amplicon comprising the gag and protease genetic sequences or a portion thereof;
characterized in that the optional reverse-transcription of the HIV RNA of step i.a) is performed with primer SEQ ID no. 1; and the amplification of the HIV DNA sequence of step ii) is performed with primers SEQ ID. No. 2-4; and provided that any one of the steps of the method for amplifying the gag and protease genetic sequences is not practised on the human or animal body.

6. A method for determining the nucleotide sequence of the gag and protease genes of a human immunodeficiency virus (HIV) comprising the sequencing of the amplicon as obtained in step ii) of claim 5, using at least 8 of the primers selected from SEQ ID. no. 5-15.

7. The nucleotide sequence of the gag and protease genes of a human immunodeficiency virus (HIV) determined by the method according to claim 6.

8. A method for the preparation of a recombinant virus comprising the amplicon as obtained in step ii) of claim 4, said method comprising the homologous recombination of the amplicon obtained in step ii) of claim 5 with a vector according to any one of claims 2-4.

9. A recombinant virus obtainable by the method according to claim 8.

10. A method for determining the phenotypic susceptibility of a human immunodeficiency virus to at least one drug, comprising the monitoring of the replicative capacity of the recombinant virus according to claim 9 in the presence of at least one cell and the at least one drug.

11. The method according to claim 10 wherein the replicative capacity of the recombinant virus is compared to the replicative capacity of an HIV virus with mutant gag and protease genetic sequences in the presence of the same at least one drug.

12. A method according to claim 11 for determining the resistance of a human immunodeficiency virus to a protease inhibitor in which the human immunodeficiency virus has a mutant gag genetic sequence.

13. A method for designing a treatment regimen for an HIV infected patient, wherein the treatment regimen is selected based on the phenotypic susceptibility determined according to any one of claims 10-11, and wherein the amplicon obtained in step ii) of claim 5—which is recombined with a vector according to any one of claims 2-4—, is obtained from the HIV RNA or DNA sequences extracted from a sample of said HIV infected patient.

14. A method for identifying a drug effective against the HIV gag and/or protease genes comprising the method according to any one of claims 10-11, wherein the at least one drug is the drug whose effectiveness is to be identified.

15. A method for determining the genotypic alterations in the HIV gag and protease nucleotide sequences comprising the comparison of the nucleotide sequence according to claim 7, with the gag and protease nucleotide sequences of a wild-type HIV virus.


SEQUENCE LISTING (19 sequences)

SEQ ID NO: 1 - DNA, 25 bp, Human immunodeficiency virus
ccattgtttaacttttgggccatcc

SEQ ID NO: 2 - DNA, 22 bp, Human immunodeficiency virus
caagtagtgtgtgcccgtctgt

SEQ ID NO: 3 - DNA, 22 bp, Human immunodeficiency virus
tggaaaatctctagcagtggcg

SEQ ID NO: 4 - DNA, 25 bp, Human immunodeficiency virus
ccattcctggctttaattttactgg

SEQ ID NO: 5 - DNA, 22 bp, Human immunodeficiency virus
tttgactagcggaggctagaag

SEQ ID NO: 6 - DNA, 20 bp, Human immunodeficiency virus
gacaagatagaggaagagca

SEQ ID NO: 7 - DNA, 20 bp, Human immunodeficiency virus
catagcaggaactactagta

SEQ ID NO: 8 - DNA, 20 bp, Human immunodeficiency virus
atgacagcatgtcagggagt

SEQ ID NO: 9 - DNA, 18 bp, Human immunodeficiency virus
attatcagaaggagccac

SEQ ID NO: 10 - DNA, 16 bp, Human immunodeficiency virus
aagacaccaaggaagc

SEQ ID NO: 11 - DNA, 20 bp, Human immunodeficiency virus
tctacatagtctctaaaggg

SEQ ID NO: 12 - DNA, 20 bp, Human immunodeficiency virus
gtggggctgttggctctggt

SEQ ID NO: 13 - DNA, 20 bp, Human immunodeficiency virus
tcttgtggggtggctccttc

SEQ ID NO: 14 - DNA, 18 bp, Human immunodeficiency virus
gataaaacctccaattcc

SEQ ID NO: 15 - DNA, 16 bp, Human immunodeficiency virus
ttatccatcttttatg

SEQ ID NO: 16 - DNA, 9559 bp, Human immunodeficiency virus
ctagagaacgctaatacatttccctactattctatcaactatagggcctcctagctaccttctttgggtcactggtttgccattttaattaaaatcaactagtagagatacattttaagaaaaacactgtatgtgtgtgtacatacacacacacacatgtatgtgtataatatacatatatatgtatgtgtataatatacatatatatgtatgtgtataatatacatatatatgtatgtgtataatatacatatatatgtatgtgtataatatacatatgtatatgtatatgtatatatgtatctgtgtatatatcttgcatttttgtaagaaaaaaacagaaaatatagaagttttcaagaactaacactttcttacataacaaagcagaaatgttcgaactacgtaactaaaatgatgaaaaaattcccagtatcactgcctgtttggtgtggctatcagaggtttattttccccctttcttgtttgctatttctttaagtcaatctggcccccatggcctctgactctgtgactcggcaccagcgctgtggccccttcatttacatttgataactgtagagagattaattataatcctgctcattagacagatcaatctgaagttggcaagtttttaaatataactacctagcatttttaaaaagggatgcctttacagtttagttaacaatatatactgcacattttgtttttaaaaggcctgtttactaccactgattaactatatacttactgaggcaactccttcttttgttttattcaaatatttactgagtaccaggactcctgtgtgctaatacaatggtgctctactttctgcacctatatactagggagaccaagcactatcacccatacctctgagagtagcttccctaactgggttactcctgagttaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagcccaattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatacagacaatggcagcaatttcaccagtgctacggttaaggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcct

SEQ ID NO: 17 - DNA, 7739 bp, Human immunodeficiency virus
cgcgccaatgaaggtgaccggaaggcgtcaattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatacagacaatggcagcaatttcaccagtgctacggttaaggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagaacgctaatacatttccctactattctatcaactatagggcctcctagctaccttctttgggtcactggtttgccattttaattaaaatcaactagtagagatacattttaagaaaaacactgtatgtgtgtgtacatacacacacacacatgtatgtgtataatatacatatatatgtatgtgtataatatacatatatatgtatgtgtataatatacatatatatgtatgtgtataatatacatatatatgtatgtgtataatatacatatgtatatgtatatgtatatatgtatctgtgtatatatcttgcatttttgtaagaaaaaaacagaaaatatagaagttttcaagaactaacactttcttacataacaaagcagaaatgttcgaactacgtaactaaaatgatgaaaaaattcccagtatcactgcctgtttggtgtggctatcagaggtttattttccccctttcttgtttgctatttctttaagtcaatctggcccccatggcctctgactctgtgactcggcaccagcgctgtggccccttcatttacatttgataactgtagagagattaattataatcctgctcattagacagatcaatctgaagttggcaagtttttaaatataactacctagcatttttaaaaagggatgcctttacagtttagttaacaatatatactgcacattttgtttttaaaaggcctgtttactaccactgattaactatatacttactgaggcaactccttcttttgttttattcaaatatttactgagtaccaggactcctgtgtgctaatacaatggtgctctactttctgcacctatatactagggagaccaagcactatcacccatacctctgagagtagcttccctaactgggttactcctgagttaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaag

SEQ ID NO: 18 - DNA, 13188 bp, Human immunodeficiency virus
tggcctctgactctgtgactcggcaccagcgctgtggccccttcatttacatttgataactgtagagagattaattataatcctgctcattagacagatcaatctgaagttggcaagtttttaaatataactacctagcatttttaaaaagggatgcctttacagtttagttaacaatatatactgcacattttgtttttaaaaggcctgtttactaccactgattaactatatacttactgaggcaactccttcttttgttttattcaaatatttactgagtaccaggactcctgtgtgctaatacaatggtgctctactttctgcacctatatactagggagaccaagcactatcacccatacctctgagagtagcttccctaactgggttactcctgagttaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgccaatgaaggtgaccggaaggcgtcaattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatacagacaatggcagcaatttcaccagtgctacggttaaggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaacgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattgacagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtccaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcggaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagaggatccccgggtaccgagctcgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttgcatgcctgcaggtcgactctagagaacgctaatacatttccctactattctatcaactatagggcctcctagctaccttctttgggtcactggtttgccattttaattaaaatcaactagtagagatacattttaagaaaaacactgtatgtgtgtgtacatacacacacacacatgtatgtgtataatatacatatatatgtatgtgtataatatacatatatatgtatgtgtataatatacatatatatgtatgtgtataatatacatatatatgtatgtgtataatatacatatgtatatgtatatgtatatatgtatctgtgtatatatcttgcatttttgtaagaaaaaaacagaaaatatagaagttttcaagaactaacactttcttacataacaaagcagaaatgttcgaactacgtaactaaaatgatgaaaaaattcccagtatcactgcctgtttggtgtggctatcagaggtttattttccccctttcttgtttgcttttctttaagtcaatctggccccca

SEQ ID NO: 19 - DNA, 9557 bp, Human immunodeficiency virus
aaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagaacgctaatacatttccctactattctatcaactatagggcctcctagctaccttctttgggtcactggtttgccattttaattaaaatcaactagtagagatacattttaagaaaaacactgtatgtgtgtgtacatacacacacacacatgtatgtgtataatatacatatatatgtatgtgtataatatacatatatatgtatgtgtataatatacatatatatgtatgtgtataatatacatatatatgtatgtgtataatatacatatgtatatgtatatgtatatatgtatctgtgtatatatcttgcatttttgtaagaaaaaaacagaaaatatagaagttttcaagaactaacactttcttacataacaaagcagaaatgttcgaactacgtaactaaaatgatgaaaaaattcccagtatcactgcctgtttggtgtggctatcagaggtttattttccccctttcttgtttgcttttctttaagtcaatctggcccccatggcctctgactctgtgactcggcaccagcgctgtggccccttcatttacatttgataactgtagagagattaattataatcctgctcattagacagatcaatctgaagttggcaagtttttaaatataactacctagcatttttaaaaagggatgcctttacagtttagttaacaatatatactgcacattttgtttttaaaaggcctgtttactaccactgattaactatatacttactgaggcaactccttcttttgttttattcaaatatttactgagtaccaggactcctgtgtgctaatacaatggtgctctactttctgcacctatatactagggagaccaagcactatcacccatacctctgagagtagcttccctaactgggttactcctgagttaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaa